(19) United States
(12) Patent Application Publication
(10) Pub. No.: US 2020/0405125 A1
(43) Pub. Date: Dec. 31, 2020

(54) MANUFACTURE OF DISTAL OPTICS

(71) Applicant: Canon U.S.A., Inc., Melville, NY (US)

(72) Inventor: Alexander Altshuler, Cambridge, MA (US)

(21) Appl. No.: 16/912,115

(22) Filed: Jun. 25, 2020

Related U.S. Application Data
(60) Provisional application No. 62/867,790, filed on Jun. 27, 2019.

(51) Int. Cl. A61B 1/00; B24B 13/00; A61B 1/07

(52) U.S. Cl. CPC A61B 1/0011; A61B 1/07; A61B 1/00096; B24B 13/0018

(57) ABSTRACT

A method of making a plurality of optical devices repeatedly slices, at a first specified angle, a first wafer of light transmissive material having a diffraction surface, to form at least one first wafer slice, and repeatedly slices, at a second specified angle, a second wafer of light transmissive material, to form at least one second wafer slice. An end portion of the second wafer slice is ground to form an interconnection surface. A plurality of illumination guides may optionally be affixed to the interconnection surface along a length of the second wafer slice, and a first wafer slice is affixed to the second wafer slice to form a sheet. The sheet is repeatedly sliced along a width of the second wafer slice to form a plurality of optical devices.

### CROSS REFERENCE TO RELATED APPLICATIONS

The present application claims the benefit of priority from U.S. Provisional Patent Application No. 62/867,790 filed Jun. 27, 2019, which is hereby incorporated by reference herein in its entirety.

### BACKGROUND

### Field of the Disclosure

The present disclosure generally relates to distal optics, and more particularly, to a method to manufacture distal optics, such as spectrally encoded endoscopy (SEE) probes or the like, and to distal optics corresponding to such methods.

### Description of the Related Art

Using optical fiber for imaging is becoming more and more prevalent in a variety of applications that can benefit from small probe size and high fidelity images. In most of these applications, to provide a reasonable field of view, a rotating probing fiber with distal focusing optics is employed. Because these probes are frequently required to be disposable in most medical applications, it is important to keep the cost of such probes as low as possible, and accordingly a method of manufacturing such probes at low cost would be extremely beneficial.

### SUMMARY

According to an aspect of the present disclosure, a method of making a plurality of optical devices, includes repeatedly slicing, at a first specified angle, a first wafer of light transmissive material having a diffraction surface, to form at least one first wafer slice, and repeatedly slicing, at a second specified angle, a second wafer of light transmissive material, to form at least one second wafer slice. An end portion of the second wafer slice is ground to form an interconnection surface. A plurality of illumination guides may be affixed to the interconnection surface along a length of the second wafer slice. A first wafer slice is affixed to the second wafer slice to form a sheet. The sheet is repeatedly sliced along a width of the second wafer slice to form a plurality of optical devices. For example, the sheet may be repeatedly sliced though the first wafer slice and the second wafer slice between affixed illumination guides to form the plurality of optical devices. Beneficially, many optical devices can be formed at roughly the same, and with a minimal of affixing operations and slicing operations, so as to minimize manufacturing costs.

Further features and aspects of the present disclosure will be apparent from the following description of example embodiments with reference to the drawings.

### BRIEF DESCRIPTION OF THE DRAWINGS

FIG. 1 is a schematic diagram of a spectrally encoded endoscopy (SEE) probe which is freed from a preassembled wafer of FIG. 11 or an assemblage of FIG. 10, by slicing through the gaps between the illumination guides in accordance with an embodiment of the present disclosure.

FIG. 2 is a flow chart of a method of making an SEE probe in accordance with an embodiment of the present disclosure.

FIG. 3 is a schematic diagram of a grating wafer in accordance with an embodiment of the present disclosure.

FIG. 4A is a schematic diagram of a grating wafer bearing a plurality of parallel disposed grating grooves and grating ridges in accordance with an embodiment of the present disclosure, and FIG. 4B is a side view of the grating wafer showing examples of the diffraction grooves and diffraction ridges in accordance with an embodiment of the present disclosure.

FIG. 5A is a schematic diagram of a grating wafer that is sliced into a plurality of grating wafer slices in accordance with an embodiment of the present disclosure, and FIG. 5B is a close-up view of a portion of FIG. 5A.

FIG. 6 is a schematic diagram of a spacer wafer in accordance with an embodiment of the present disclosure.

FIG. 7 is a schematic diagram of a spacer wafer that is sliced into a plurality of spacer wafer slices in accordance with an embodiment of the present disclosure.

FIG. 8 is a schematic diagram of a plurality of spacer wafer slices that are assembled together and rotated so that the end portions are exposed to facilitate their removal in accordance with an embodiment of the present disclosure.

FIG. 9 is a schematic diagram of an assemblage of spacer wafer slices as in FIG. 8 that is ground to remove the exposed end portions in accordance with an embodiment of the present disclosure.

FIG. 10 is a schematic diagram of a spacer wafer slice that has been ground to form an interconnection surface and of a plurality of illumination guides that are affixed to the spacer wafer slice along a length direction of the interconnection surface in accordance with an embodiment of the present disclosure.

FIG. 11 is a schematic diagram of a spacer wafer slice, that is ground and affixed to a plurality of illumination guides, and to which a grating wafer slice is affixed in accordance with an embodiment of the present disclosure.

FIG. 12 is a schematic diagram of a plurality of the assemblages of FIG. 11 that are stacked atop one another to facilitate the freeing of individual SEE probes with a minimal number of slicing operations in accordance with an embodiment of the present disclosure.

FIG. 13 is a block diagram of an apparatus in accordance with an embodiment of the present disclosure.

### DESCRIPTION OF THE EMBODIMENTS

Various exemplary embodiments, features, and aspects of the present disclosure are described in detail below with reference to the drawings.

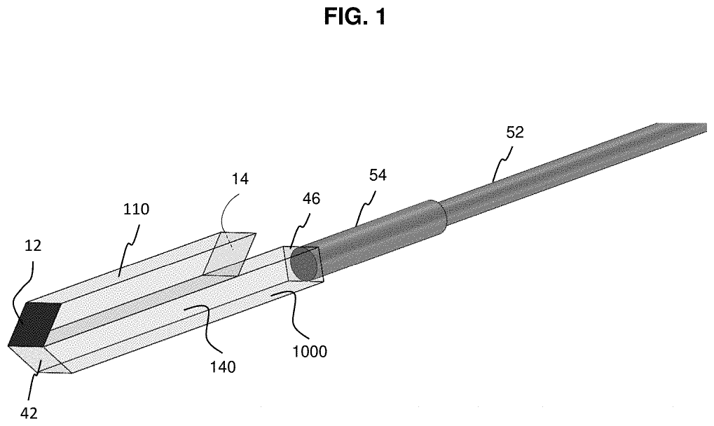

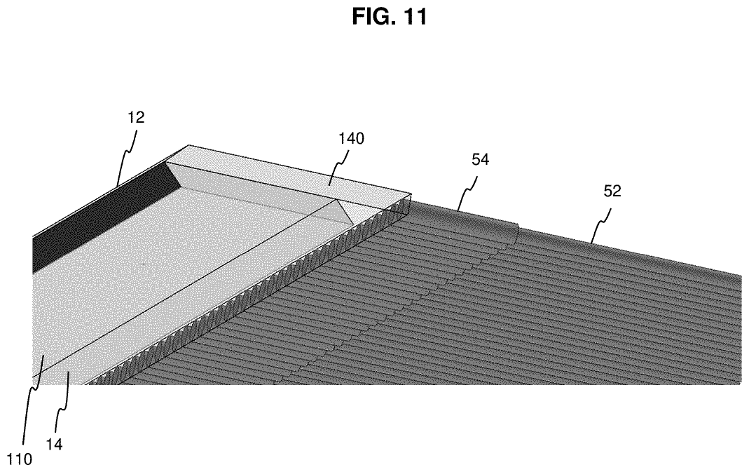

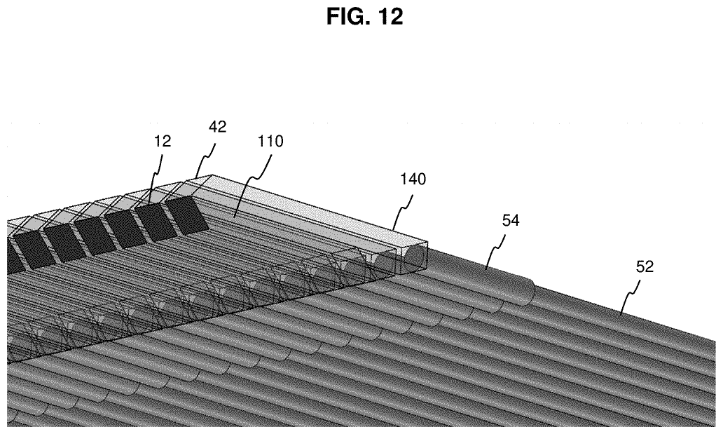

Referring now to FIG. 1, there is shown an exemplary spectrally encoded endoscopy (SEE) probe 1000 which is freed from a preassembled wafer of FIG. 12 or an assemblage of FIG. 11, by slicing through the gaps between the illumination guides, in accordance with an aspect of the present disclosure.

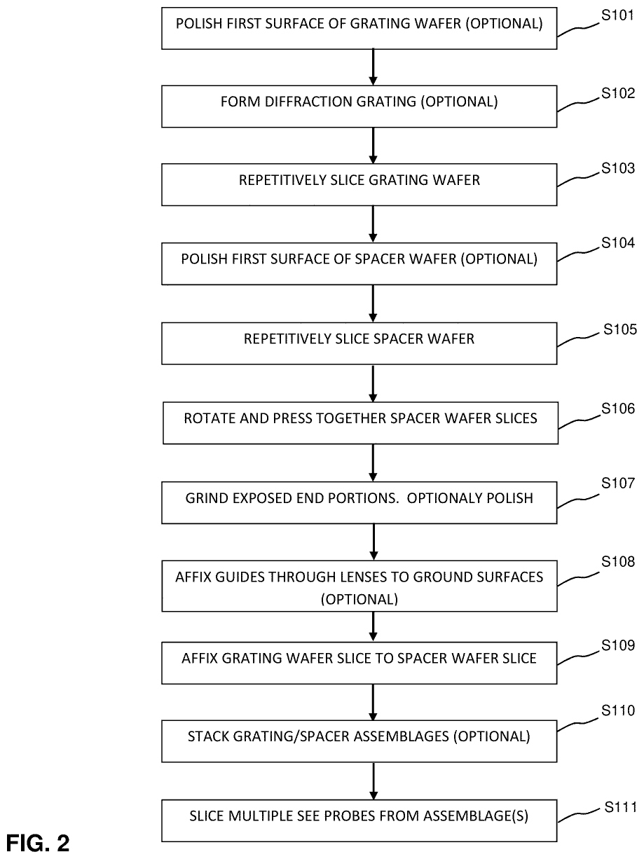

FIG. 2 shows a flow chart of a method of making an SEE probe in accordance with the first embodiment of the present disclosure. The first embodiment of the method can begin at step S101 wherein if the first surface 12 of the grating wafer 10 is not already sufficiently smooth that a diffraction grating can be formed thereon, the first surface 12 is polished to provide such a smooth surface. Processing the first surface 12 using a conventional single-sided polishing technique is sufficient for providing the smooth surface. For example, conventional lapping of the first surface 12 using either a hard lap or a soft metal lap, and additionally using a conventional polishing paste or polishing suspension, may be used. Alternatively, the first surface 12 may be polished using a soft fabric or composite and a conventional polishing paste or polishing suspension. Step S101 is considered an optional step, as, for example, already polished (or otherwise suitably smooth) optical glass wafers are readily available from numerous commercial sources. If the grating wafer 10 already has a smooth first surface 12, then the first embodiment of the method can begin at step S102 (discussed below).

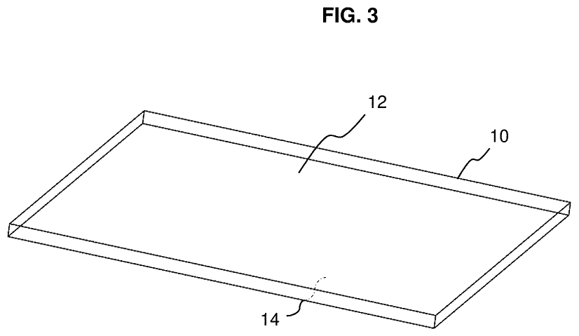

FIG. 3 is a schematic diagram of grating wafer 10 of a first embodiment to make an SEE probe, in accordance with an aspect of the present disclosure. The grating wafer 10 has a first surface 12 and has a second surface 14 opposite to the first surface 12. That is, the second surface 14 is on the opposite side of the grating wafer 10 as the first surface 12. The grating wafer 10 is composed of a light transmissive material. For example, the grating wafer 10 may be composed of glass, heat-curable resin, UV-curable resin, plastic, ceramic, or a combination thereof, and is transparent, or at least light transmissive, over a specified spectrum of electromagnetic radiation, such as microwave radiation, infrared light, visible light, ultraviolet light, and/or broadband radiation over a variety of one or more of the above wave bands. In FIG. 3, the grating wafer 10 does not yet bear a diffraction grating (or other diffraction surface), and moreover, the first surface 12 may be either polished or unpolished. Conventional optical glass provides a high degree of light transmissivity and a high degree of structural stability at a low cost, and if processed in accordance with an embodiment of the present disclosure, is well suited as a structural material for making quality SEE probes at low cost. Accordingly, in the following discussion, except where noted, it is assumed that the grating wafer 10 is composed of conventional optical glass.

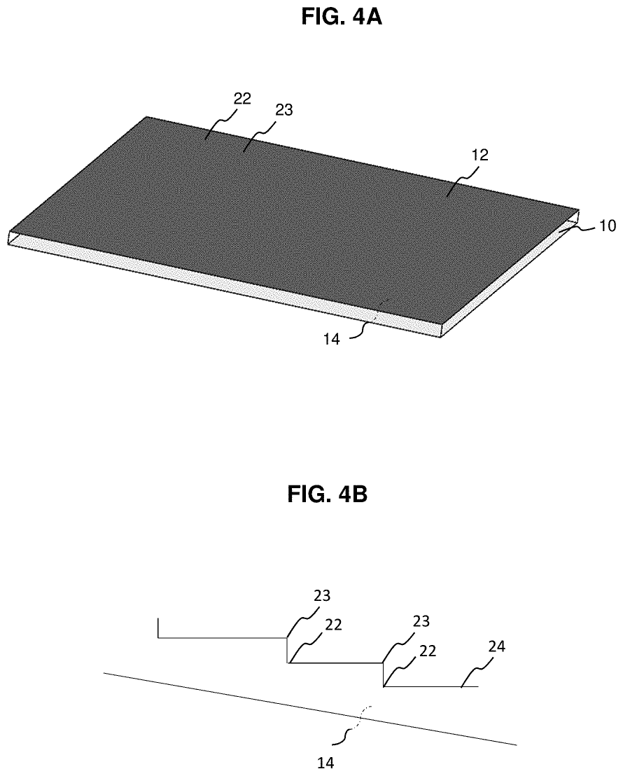

Referring to FIG. 4A, there is shown a schematic diagram of the grating wafer 10 which bears a diffraction grating 24 formed in or on grating wafer 10 at the first surface 12. Referring to FIG. 4B, there is shown a side view of the grating wafer 10 showing that the diffraction grating 24 comprises a plurality of diffraction grooves 22 parallel to one another and interposed by a plurality of diffraction ridges 23 which are parallel to one another. It is not essential to use such a diffraction grating to provide diffraction. For example, in alternative embodiments of the present disclosure, other forms of diffraction grating 24 are alternatively used, such as a fractal diffraction grating, or alternatively a pattern of Fresnel diffraction windows may be used to provide diffraction. As another example, in an alternative embodiment, one or more Fresnel diffraction windows may be formed by first leaving the first surface 12 unpolished (or by tinting the first surface) and then second polishing the unpolished or tinted first surface at each target window site to provide the pattern of Fresnel diffraction windows. A wide variety of diffraction gratings or windows may be used. However, as a diffraction grating performs well and is relatively inexpensive to manufacture, it is assumed in this embodiment (except where noted below) that the diffraction grating 24 comprises a plurality of straight line parallel diffraction grooves 22 interposed by a corresponding plurality of straight line parallel diffraction ridges 23.

In accordance with the first embodiment, the diffraction grating 24 is formed in step S102 by etching the first surface 12 of the grating wafer 10 using a hard scribe under machine control. The scribe is preferably sufficiently hard that etched lines of the etching are of a consistent shape throughout their respective lengths and moreover between the different etched lines. For example, a computer numerical control (CNC) machining apparatus may be used to etch a diamond scribe into the first surface 12 to form the diffraction grooves 22. Alternatively, a conventional CNC apparatus may be used to control an abrasive water jet to cut or mill the diffraction grooves 22 into the first surface 12. In the first embodiment, the formation of the diffraction grooves 22 likewise forms the diffraction ridges 23 interposed therewith. Moreover, the polished or otherwise smooth first surface 12 remains at tip portions of the diffraction ridges 23, and the tip portions of the diffraction ridges 23 are accordingly roughly coplanar by virtue of the smoothness provided to the first surface 12 of the grating wafer 10 prior to the formation of the diffraction grating 24 (e.g. in a case wherein the grating wafer 10 is polished as discussed above, upon the completion of such polishing).

As another alternative embodiment, the diffraction grooves 22 may be composed, for example, parallel concentric grooves such as parallel concentric circular grooves or parallel concentric elliptical grooves, and likewise, the diffraction ridges 23 may be composed, for example, of parallel concentric ridges such as parallel concentric circular ridges or parallel concentric elliptical ridges. Other shapes of grooves and/or ridges are also in accordance with the present embodiment. Moreover, the shapes of the diffraction grooves 22 and the diffraction ridges 23 may be selected using conventional design rules and/or a conventional method so as to provide a selected diffraction pattern.

Although the diffraction grooves 22 may be cut into the first surface 12 of the grating wafer 10, for example, as discussed above, it is also possible to mold the diffraction grooves 22 and the diffraction ridges 23 into the first surface 12 using a mold. For example, the grating wafer 10 may be heated and then pressed into a diffraction pattern mold. Alternatively, suitable diffraction grooves 22 and the corresponding diffraction ridges 23, may be provided by forming the grating wafer 10 in a mold (not shown) that has a pattern for forming the diffraction grooves and/or diffraction ridges, and this variation of the embodiment is highly convenient when the grating wafer 10 is to be formed of heat-curable resin, UV-curable resin, plastic, and/or ceramic. As yet another alternative, the diffraction ridges 23 may be printed onto the first surface 12 of the grating wafer 10, for example, using a micro-imprint process. As another alternative, a grating wafer 10 (or other form of diffraction wafer) may be obtained from a commercial source, in which case the first embodiment of the method can begin at step S103 (discussed below).

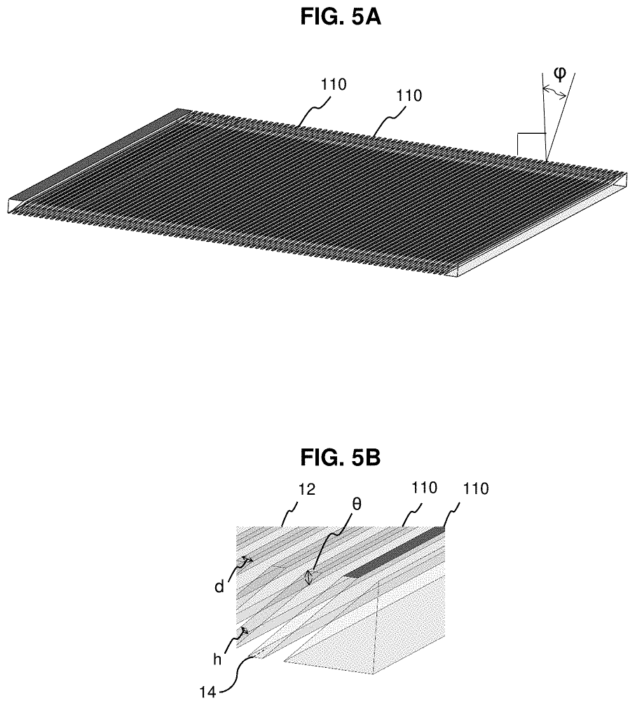

Referring to FIG. 5A, there is shown a schematic diagram of the grating wafer 10 that is sliced into a plurality of grating wafer slices 110, and a close-up view of a portion of the wafer slices 110 is shown in FIG. 5B. Each grating wafer slice 110 has a first surface 12, a second surface 14, a plurality of diffraction grooves 22, and a plurality of diffraction ridges 23 which are inherited from the grating wafer 10 (from which the grating wafer slice 110 is sliced). In accordance with the first embodiment of the present disclosure, the grating wafer 10 bearing the diffraction grating 24 (or other diffraction component) is repeatedly sliced in step S103 at a slice angle θ relative to a tangent of the first surface 12 to form a plurality of grating wafer slices 110. Each grating wafer slice 110 has a cut edge at an acute angle θ relative to the first surface 12 and has another cut edge at an obtuse angle π-θ relative to the first surface 12. To perform the slicing, a CNC machining apparatus may be set to provide a cutting angle φ relative to a normal to the first surface 12 to provide the slice angle θ (the acute angle between one of the cut edges of a grating wafer slice 110 and the first surface 12) of the grating wafer slice 110 by using Expression 1 provided below,

φ=π/2−0 (Expression 1)

wherein φ, θ, and π/2 are each expressed in radians. Note that the obtuse angle π-θ can alternatively be obtained using Expression 2 provided below,

π−θ=π/2+φ (Expression 2)

wherein π, φ, θ, and π/2 are each expressed in radians.

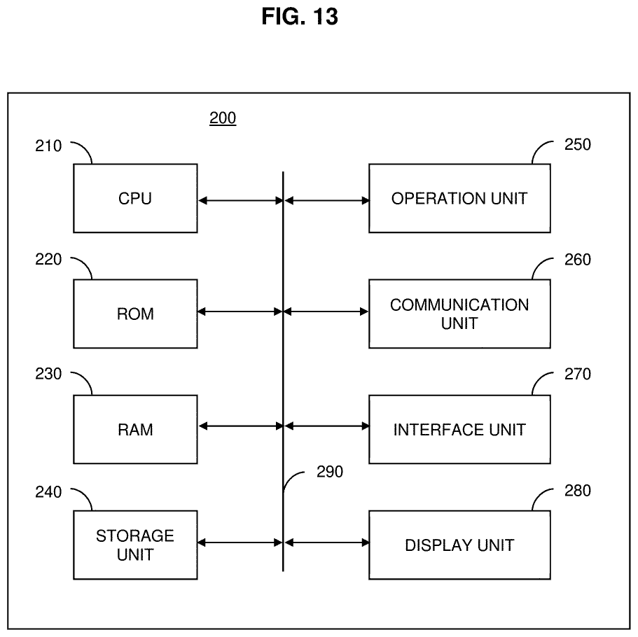

In this embodiment, the grating wafer slices 110 are sliced out of the grating wafer 10 along the same direction as the parallel diffraction grooves 22 and diffraction ridges 23. The first surface 12 of each grating wafer slice 110 includes a sufficient number of the diffraction ridges 23 to provide a suitable diffraction pattern when excited by a specified bandwidth (or specified bandwidths) of illumination light. In this embodiment, once assembled as the SEE probe 1000 of FIG. 13, in operation, the illumination light is provided to a grating wafer slice 110 through a surface other than the first surface 12, and exits the grating wafer slice 110 at the first surface 12 through the diffraction grating 24, which diffracts the imaging light onto an imaging target. The specified bandwidth or bandwidths can be, for example, user provided input(s).

In accordance with the embodiment, a thickness h of each grating wafer slice 110 is a design input parameter that is specified by a user in accordance to the specific usage of the SEE probe 1000. Accordingly, a distance between to provide between each grating wafer slice 110 is a function of the thickness h. As the grating wafer slices 110 are sliced off at the angle φ relative to a normal to the first surface 12, the distance d between grating wafer slices 110 is not equal to h. The distance d can be obtained using Expression 3 provided below,

d=h/sin θ (Expression 3)

wherein d is the distance between grating wafer slices 110 along the first surface 12 and h is the thickness of each grating wafer slice 110. The foregoing is based on an assumption that material lost through the slicing process is negligible. Additional distance can be added between each slice to account for such lost material, or alternatively, to account for the thickness of a diamond saw, water jet, or the like, based on the particulars of each such slicing/cutting apparatus. When such additional distance is provided, the above-provided Expression 3 remains valid except that d is the measure between the cut surfaces of a grating wafer slice 110 along the first surface 12.

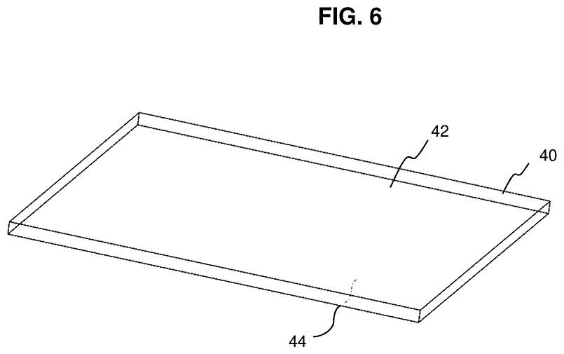

Referring to FIG. 6, there is shown a schematic diagram of a spacer wafer 40 in accordance with the first embodiment of the present disclosure. The spacer wafer 40 has a first surface 42 and has a second surface 44 opposite to the first surface 42. The spacer wafer 40 is composed of a light transmissive material. For example, the spacer wafer 40 may be composed of glass, heat-curable resin, UV-curable resin, plastic, ceramic, or a combination thereof, and is transparent, or at least light transmissive, over a specified spectrum of electromagnetic radiation, such as microwave radiation, infrared light, visible light, ultraviolet light, and/or broadband radiation over a variety of one or more of the above wave bands. For example, the spacer wafer 40 may be formed of the same material as the grating wafer 10, and may be either transparent or light transmissive over the same specified spectrum as the grating wafer 10. It can be beneficial to make the spacer wafer 40 from the same (or similar) material as the grating wafer 10, for example, to facilitate bonding of the spacer wafer 40 to the grating wafer 10 as discussed below. For example, it can beneficial for the spacer wafer 40 to have the same (or a similar) coefficient of thermal expansion as the grating wafer 10 to avoid warping of the SEE probe 1000, for example due to the heat of a patient's body, and a potential for a slight reduction in efficiency or imaging quality due to such warping. Moreover, as the spacer wafer 40 and grating wafer 10 will be bonded together (as discussed below), having the aforementioned same (or similar) coefficient of thermal expansion reduces thermal stress on the bond, and accordingly, reduces the cost of providing a sufficiently strong bond. The specified bandwidth or bandwidths of light transmissivity for the spacer wafer can be, for example, user provided input(s).

In step S104, if the first surface 42 of the spacer wafer 40 is not already sufficiently smooth to provide a mirror like surface at low angles of incidence, the first surface 42 is polished to provide such a smooth surface. Processing the first surface 42 using a conventional single-sided polishing technique is sufficient for providing the smooth surface. Like the first surface 12 of the grating wafer 10, the first surface 42 of the spacer wafer 40 can be lap polished using either a hard lap or a soft metal lap, and additionally, a conventional polishing paste or polishing suspension. Alternatively, the first surface 42 may be polished using a soft fabric or composite and a conventional polishing paste or polishing suspension. Step S104 is considered an optional step, as for example already polished (or otherwise suitably smooth) optical glass wafers are readily available from numerous commercial sources.

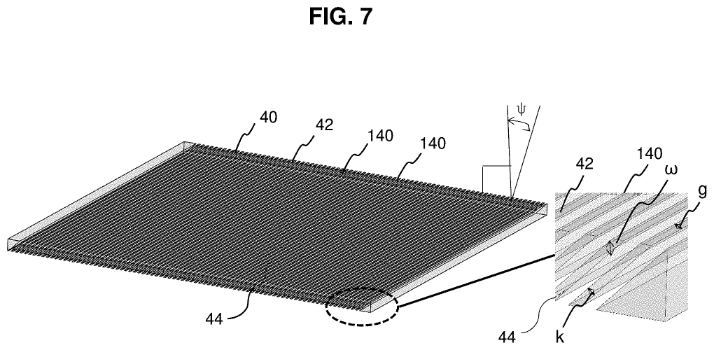

Referring now also to FIG. 7, there is shown a schematic diagram of a spacer wafer 40 that is sliced in step S105 into a plurality of spacer wafer slices 140 in accordance with the first embodiment of the present disclosure. Each spacer wafer slice 140 has a first surface 42 and a second surface 44 which are inherited from the spacer wafer 40 (from which the spacer wafer slice 140 is sliced). The slicing may be performed, for example, using conventional CNC dicing or conventional CNC abrasive water jet cutting. For example, to perform the slicing, a CNC machining apparatus may be set to provide a cutting angle ψ relative to a normal to the first surface 42 to provide the slice angle ω (the acute angle between one of the cut edges of a spacer wafer slice 140 and the first surface 42) of the spacer wafer slice 140 by using Expression 4 provided below,

ψ=π/2−ω (Expression 4)

wherein ψ, ψ, and π/2 are each expressed in radians. This slicing of the spacer wafer 40 is accordingly similar to the cutting of the grating wafer 10 discussed above, except note that the respective slice angles θ and ω (and thus the respective cutting angles φ and ψ) do not need to be equal. Rather, the respective slice angles θ and ω are set to provide an appropriate angle of illumination of the diffraction grating (and thus an appropriate diffraction pattern), as discussed below. Similar to the grating wafer slicing, each spacer wafer slice 140 has a cut edge at an acute angle ψ relative to the first surface 42 and has another cut edge at an obtuse angle π−ω relative to the first surface 42. Note that the obtuse angle π−ω can alternatively be obtained using Expression 5 provided below,

π−ω=π/2+ψ (Expression 5)

wherein π, ψ, ω, and π/2 are all expressed in radians.

Similar to the grating wafer slices, the spacer wafer 40 is repeatedly sliced in step S105 to provide the plurality of spacer wafer slices 140. The thickness k of each spacer wafer slice 140 is a design input parameter. Accordingly, a distance g between each spacer wafer slice 140 along the first surface 42, (more particularly a measure between cut surfaces of the spacer wafer slice 140 along the first surface 42) can be obtained by using Expression 6 provided below,

g=k/sin ω (Expression 6)

wherein k is design input parameter value that can be user specified, for example.

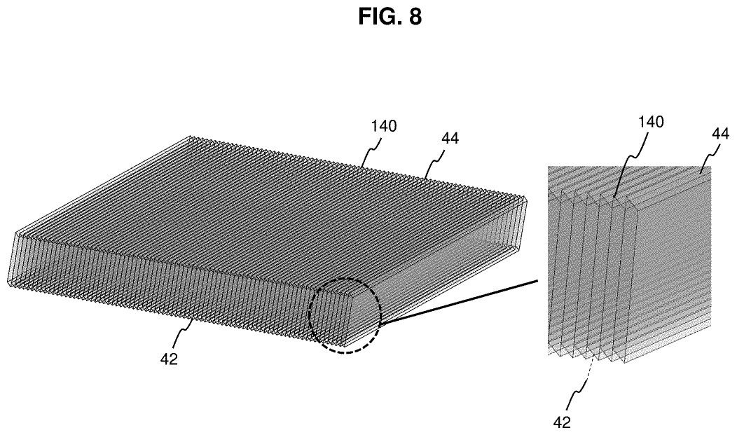

Referring to FIG. 8, there is shown a schematic diagram of the spacer wafer slices 140 that are assembled (e.g. pressed together), flipped over, and rotated through an angle of ψ radians. Note that in FIG. 8, the spacer wafer slices are shown flipped over (relative to FIG. 5) so that the respective first surface 42 of each spacer wafer slice 140 is shown on the downward side in FIG. 8, thus the respective second surface 44 of each spacer wafer slice 140 is shown on the upward side in FIG. 8. The spacer wafer slices 140 are pressed together in step S106. Light pressure is sufficient in the assembling S106, as once the spacer wafer slices 140 are placed into contact with one another by pressing, any movement tending to pull the wafer spacer wafer slices 140 apart would induce a slight vacuum between the spacer wafer slices 140 which would in turn tend to hold the spacer wafer slices 140 together. Moreover, in step S106 the spacer wafer slices 140 are also rotated through an angle of ψ radians so that the end portions on the second surface 44 are exposed. In particular, in FIG. 8, the spacer wafer slices 140 are also rotated in step S106 through an angle of ψ radians in a counterclockwise direction so that the cut edges of the spacer wafer slices 140 are arranged vertically, and accordingly, so that the end portions on the second surface 44 are exposed at the top of the illustration of the spacer wafer slices 140 in FIG. 8. This facilitates a cost effective processing of the spacer wafer slices 140 discussed next. Note that it is not essential to rotate the spacer wafer slices 140 by exactly ψ radians, as discussed below.

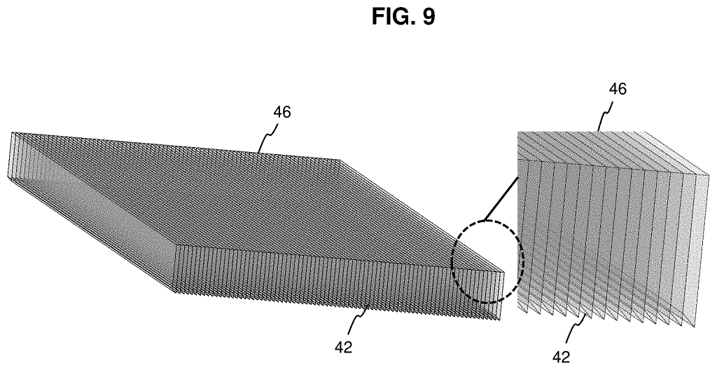

Referring to FIG. 9, there is shown a schematic diagram of the assemblage of the spacer wafer slices 140 (of FIG. 8) which are ground in step S107 on the second surface 44 to remove the exposed end portions. In particular, in step S107, the end portions of the second surfaces 44 of the spacer wafer slices 140 that were exposed by the rotation provided in step S106 are now ground along a horizontal direction until the ground surface 46 of each spacer wafer slice 140 is perpendicular (or roughly perpendicular) to the cut surfaces of that spacer wafer slice 140. The ground surfaces 46 of the spacer wafer slices 140 may also optionally be polished in step S107 after the grinding (for example using lap polishing as discussed above) to provide smooth light transmissive surface suitable for bonding to a lens (as discussed below). As an alternative, the flipping operation of the spacer wafer slices 140 discussed with respect to FIG. 8 can be omitted, in which case, for example, the second surfaces 44 of the assembled spacer wafer slices 140 can be ground in step S107 from below. Note that during the grinding and the optional polishing, the pressure applied to the spacer wafer slices 140 should be provided at a sufficient level (for example using a chuck) to prevent their movement relative to one another.

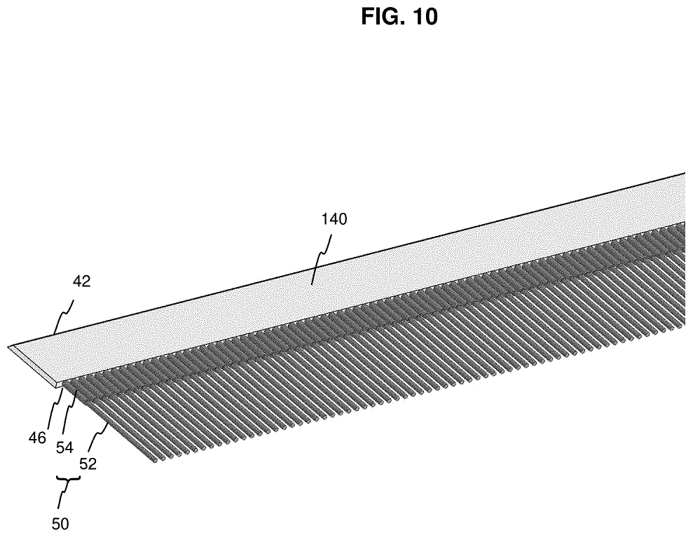

Referring to FIG. 10, there is shown a schematic diagram of a spacer wafer slice 140 that has been removed from the assemblage of spacer wafer slices 140 of FIG. 9 after the grinding and any optional polishing. Also shown in FIG. 8 a plurality of optional illumination light guides 50. In this embodiment, each illumination light guide 50 includes both a transmission guide 52 and an interconnection lens 54. The transmission guide 52 and the interconnection lens 54 may be composed of glass, heat-curable resin, UV-curable resin, plastic, ceramic, or a combination thereof, and is transparent, or at least light transmissive, over a specified spectrum of electromagnetic radiation, such as microwave radiation, infrared light, visible light, ultraviolet light, and/or broadband radiation over a variety of one or more of the above wave bands, and this specified spectrum may, for example, be the same as the specified bandwidth of the spacer wafer 40. The specified bandwidth or bandwidths of the illumination light guides 50 can be, for example, user provided input(s).

As an example, the transmission guide 52 may be either a single-mode optical fiber or a multi-mode optical fiber, and the interconnection lens 54 may be a GRIN (gradient index) lens. In each illumination light guide 50, the transmission guide 52 may optionally be affixed in optional step S108 at one end to an end of the interconnection lens 54, and moreover, the other end of each interconnection lens 54 may optionally be affixed in step S108 to the ground surface 46 of the spacer wafer slice 140. For example, the illumination light guides may be arranged side-to-side (with a small gap in between) in a line along a length direction of the spacer wafer slice 140, and each interconnection lens 54 may be affixed to the ground surface 46 of the spacer wafer slice 140 over a respective distinct portion of the length of the spacer wafer slice 140. Moreover, the respective small gap between adjacent illumination light guides 50 provides a corresponding portion of unoccupied space (gap) the ground surface 46 of the spacer wafer slice 140. The unoccupied space is sufficiently large that the cutting apparatus can cut through each of the gaps to separate the respective illumination light guides 50 at a later processing step. The affixing of the transmission guide 52, the interconnection lens 54, and the spacer wafer slice 140 together as discussed above can be performed, for example, using a light transmissive adhesive. Alternatively, especially useful for plastic and resin components, placing the components 52, 54, and 140 into contact with one another under heat and pressure, the heat and pressure being used to affix the components to one another. As another alternative, the illumination light guides 50 may be omitted by not performing optional step S108, and if so, in operation light may be applied directly to the ground surface 46 of the spacer wafer slice 140, for example, as discussed more fully below.

Referring to FIG. 11, there is shown a schematic diagram of a spacer wafer slice 140 affixed to the illumination guides as in FIG. 10. Also shown in FIG. 11 is a grating wafer slice 110. In step S109, the grating wafer slice 110 is affixed to the spacer wafer slice 140, for example, using a light transmissive adhesive. In particular, a cut surface of the grating wafer slice 110 is affixed in step S109 to a cut surface of the spacer wafer slice 140 in the orientation that the diffraction grating 24 of the grating wafer slice 110 is adjacent to the first surface 42 of the spacer wafer slice 140, with the diffraction grating 24 and the first surface 42 forming a V-shape, in particular a bottom of a V-shape in this embodiment. In an alternative embodiment, heat and pressure can be used to affix the grating wafer slice 110 to the spacer wafer slice 140. In FIG. 11, the grating wafer slice 110 is shown as being below the spacer wafer slice 140.

Referring to FIG. 12, there is shown a schematic diagram of processing of an optional step S110 wherein a plurality of the assemblages of FIG. 9 are assembled one atop of another to form a wafer. A natural vacuum that would exist between the assemblages merely by being brought into contact with one another can be used to hold the wafer together, although some additional pressure (such as may be provided using a chuck) is useful to prevent slippage. Step S110 facilitates rapid removal of individual SEE probes by reducing the number of cutting operations used in further processing described below.

Referring back to FIG. 1, in step S111, the preassembled wafer of FIG. 12 or the assemblage of FIG. 11 is sliced into a multitude of individual SEE probes 1000 by slicing through the above-discussed unoccupied space (gap) at the ground surface 46 of the spacer wafer slice 140 disposed between respective illumination light guides 50. That is, the small gap between the illumination light guides 50 (discussed above with respect to FIG. 9) provides a region through which the grating wafer slice 110 and the spacer wafer slice 140 can be cut without disturbing the respective material of the spacer wafer slice 140 that is affixed to the respective illumination light guides 50. The slicing of step S111 thus frees a large plurality of SEE probes 1000 from one another. An individual SEE probe 1000 is a specimen or member of the large plurality of SEE probes 1000, and each such specimen SEE probe 1000 may have like size and optical characteristics as the other such specimens of the plurality of SEE probes 1000, or alternatively, the specimens can have different characteristics from one another, for example, by altering the spacing (or spacings) between cuts and/or the optical characteristics of the grating wafer 10, and/or of the spacer wafer 40, and/or of the grating wafer slices 110, and/or of the spacer wafer slices 140, over their lengths, widths, and/or heights. In the case that the preassemble wafer of FIG. 10 is so cut, corresponding multiple of layers of such large pluralities of SEE probes 1000 are freed from one another. Beneficially, in either case, a very large number of SEE probes 1000 is provided with a relatively small number of processing steps. The numbers of affixing operations and slicing operations is very small relative to the very large number of SEE probes 1000 so provided. Moreover, the assemblages of components described above assist in holding the various components in proper orientation both during affixing steps and slicing steps, which in turn improves the quality of the SEE probes 1000 while providing a large number of such probes SEE 1000 with a minimal number slicing and affixing operations. Accordingly, the above-described embodiments of the present disclosure are highly useful for reducing manufacturing costs and increasing the quality of the SEE probes 1000.

Referring again to optional step S108, if step S108 is omitted, then the SEE probes 1000 lack an affixed illumination light guide 50, and if so, in operation of such SEE probes 1000, light may be applied directly to the ground surface 46 of the spacer wafer slice 140 portion of the SEE probe 1000. As yet another alternative, some portion of the ground surface 46 of the spacer wafer slice 140 may have illumination light guides 50 affixed thereto, whereas at another portion (or portions) of the ground surface 46 of the spacer wafer slice 140 the illumination light guides 50 may be omitted. For example, illumination light guides 50 may be affixed to a central section of the ground surface 46 of one or more of the spacer wafer slices 140, whereas at one or more of the end sections of the ground surfaces 46 of the spacer wafer slices 140 the illumination light guides 50 may be omitted. This is beneficial, for example, for fully utilizing such end sections of the ground surfaces 46 of the spacer wafer slices 140 which are too short facilitate connection of an illumination light guide 50, or to ease handling of the spacer wafer slices 140 by leaving the end sections free of the illumination light guides 50.

There are various alternative embodiments of the present disclosure. For example, in accordance with an aspect of the present disclosure, slicing of the grating wafer 10 and spacing wafer 30 may be performed, for example, by conventional dicing, conventional shearing, CNC diamond sawing, CNC abrasive water jet cutting, or otherwise cutting the wafer along a line or other user specified path.

As another alternative embodiment, a spacer wafer segment can be formed in a mold so as to include both a spacer wafer slice like portion and a plurality of interconnection lenses. This eliminates the burden of affixing a large number of interconnection lenses to a spacer wafer slice 140, and accordingly, can reduce fabrication costs. However, the uniformity may suffer to some extent due to tolerances of the molding process. Nonetheless such can be sufficient where lower quality SEE optics may be acceptable, for example, in veterinary applications or the like.

The present disclosure may also be carried out in a manner in which an arrangement such as an apparatus, system, or the like, may be provided with a program for performing one or more functions according to one or more aspects of the present disclosure via a network or a non-transitory storage medium, and one or more processors of a computerized configuration(s) of the apparatus or system may read and execute the program. The present disclosure may also be carried out by circuitry (for example, an ASIC or the like) for performing one or more functions.

FIG. 13, for example, illustrates a hardware configuration of an apparatus to facilitate the techniques described below to make a plurality of optical devices according to an embodiment of the present disclosure. The apparatus may include one or more configurational components including, for example, a central processing unit (CPU) 210, a read only memory (ROM) 220, a random-access memory (RAM) 230, a storage unit 240, an operation unit 250, a communication unit 260, an interface unit 270, and a display unit 280. These components may be connected together by a bus 290 so that the components can communicate with each other. The bus 290 may be used to transmit and receive data between these pieces of hardware connected together, or transmit a command from the CPU 210 to the other pieces of hardware.

While the present disclosure has been described with reference to exemplary embodiments, it is to be understood that these exemplary embodiments are not seen to be limiting. The scope of the following claims is to be accorded the broadest interpretation so as to encompass all such modifications and equivalent structures and functions.

What is claimed is:

1. A method of making a plurality of optical devices, the method comprising:
repeatedly slicing, at a first specified angle, a first wafer of light transmissive material having a diffraction surface, to form at least one first wafer slice;
repeatedly slicing, at a second specified angle, a second wafer of light transmissive material, to form at least one second wafer slice;
grinding an end portion of the second wafer slice to form an interconnection surface;
affixing the first wafer slice to the second wafer slice to form a sheet; and
repeatedly slicing through the sheet along a width of the second wafer slice to form a plurality of optical devices.

2. The method of making a plurality of optical devices according to claim 1, wherein grinding the end portion of the second wafer forms the interconnection surface at roughly at a right angle relative to sliced surfaces of the second wafer slice.

3. The method of making a plurality of optical devices according to claim 1, further comprising affixing a plurality of illumination guides to the interconnection surface along a length of the second wafer slice.

4. The method of making a plurality of optical devices according to claim 3,
wherein at least one of the illumination guides includes an optical fiber and an interconnection lens, with an end of the optical fiber affixed to the interconnection lens, and
wherein affixing the plurality of illumination guides to the interconnection surface along a length of the second wafer slice comprises affixing the interconnection lens to the interconnection surface.

5. The method of making a plurality of optical devices according to claim 4, wherein the interconnection lens is a GRIN (gradient index) lens.

6. The method of making a plurality of optical devices according to claim 1, wherein the diffraction surface of the first wafer of light transmissive material is formed by forming a diffraction grating on a surface of a wafer of light transmissive material.

7. The method of making a plurality of optical devices according to claim 1, wherein the diffraction surface of the first wafer of light transmissive material is formed by inscribing a plurality of parallel lines into a surface of a wafer of light transmissive material to form a diffraction grating.

8. The method of making a plurality of optical devices according to claim 1,
wherein the diffraction surface of the first wafer of light transmissive material is formed by inscribing a plurality of parallel channels into a surface of a wafer of light transmissive material to form a diffraction grating, and
wherein at least one first wafer slice is formed by slicing the first wafer along a direction parallel to the plurality of parallel channels.

9. The method of making a plurality of optical devices according to claim 3, wherein affixing the plurality of illumination guides to the interconnection surface along a length of the second wafer slice comprises providing a gap between each of the illumination guides at the interconnection surface.

10. The method of making a plurality of optical devices according to claim 9, wherein repeatedly slicing through the sheet along a width of the second wafer slice to form the plurality of optical devices comprises slicing through the second wafer slice immediately adjacent the gaps so each optical device is connected to a respective illumination guide.

11. The method of making a plurality of optical devices according to claim 3, further comprising:
layering the plurality of sheets in alignment so that the illumination guides line up in a given direction.

12. The method of making a plurality of optical devices according to claim 1, wherein the optical devices are SEE (spectrally encoded endoscopy) probe illumination optics.

13. A specimen optical device made by a method of making a plurality of optical devices, the method comprising:
repeatedly slicing, at a first specified angle, a first wafer of light transmissive material having a diffraction surface, to form at least one first wafer slice;
repeatedly slicing, at a second specified angle, a second wafer of light transmissive material, to form at least one second wafer slice;
grinding an end portion of the second wafer slice to form an interconnection surface;
affixing a first wafer slice to the second wafer slice to form a sheet; and
repeatedly slicing through the sheet along a width of the second wafer slice to form a plurality of optical devices.

14. An apparatus to make a plurality of optical devices, the apparatus comprising:
one or more memories and one or more processors configured to cause the apparatus to:
repeatedly slice, at a first specified angle, a first wafer of light transmissive material having a diffraction surface, to form at least one first wafer slice;
repeatedly slice, at a second specified angle, a second wafer of light transmissive material, to form at least one second wafer slice;
grind an end portion of the second wafer slice to form an interconnection surface;
affix a first wafer slice to the second wafer slice to form a sheet; and
repeatedly slice through the sheet along a width of the second wafer slice to form a plurality of optical devices.
